(19) United States
(12) Patent Application Publication
(10) Pub. No.: US 2025/0027051 A1
(43) Pub. Date: Jan. 23, 2025

(54) CLONAL CHINESE HAMSTER OVARY CELLS AND THEIR USE

(71) Applicant: MEDIMMUNE, LLC, Gaithersburg, MD (US)

(72) Inventors: Lina CHAKRABARTI, Gaithersburg, MD (US); Raghothama CHAERKADY, Gaithersburg, MD (US)

(21) Appl. No.: 18/714,915

(22) PCT Filed: Nov. 30, 2022
(86) PCT No.: PCT/IB2022/061605

§ 371 Date: May 30, 2024

Related U.S. Application Data
(60) Provisional application No. 63/264,781, filed on Dec. 1, 2021.

(51) Int. Cl. C12N 5/071; C07K 16/00

(52) U.S. Cl. CPC C12N 5/0682; C07K 16/00; C12N 2510/00

(57) ABSTRACT

Provided herein are clonal Chinese Hamster Ovary (CHO) cells and their use in increasing cellular productivity in biomanufacturing processes. Specifically, the disclosure provides a method of improving specific cellular productivity of a polypeptide of interest in a recombinant cell comprising expressing a nucleic acid encoding the polypeptide of interest in a recombinant cell having a high mitochondrial membrane potential (MMP).

### CROSS-REFERENCE TO RELATED APPLICATIONS

This application, filed herewith on Nov. 30, 2022 claims benefit under 35 U.S.C. § 119 (e) of the following U.S. Provisional Application No. 63/264,781 filed Dec. 1, 2021. Each of the above listed applications is incorporated by reference herein in its entirety for all purposes.

### BACKGROUND OF THE DISCLOSURE

Improving recombinant protein manufacturing using Chinese hamster ovary (CHO) host cells is a major challenge for biopharmaceutical companies. The phenotypic and genotypic variations within the CHO host population are known to contribute to their versatility in growth, metabolism and biosynthetic capacity. (Davies, S. L. et al. Biotechnol Bioeng 110, 260-274. (2013); O'Callaghan, P. M. et al. Biotechol Prog 31, (2015)). The host cell heterogeneity together with random integration of expression vectors into the host genome result in recombinant cell lines that express proteins with varying productivity and product quality profiles. Therefore, development of cell lines for biotherapeutic protein production requires extensive screening of heterogenous transfectant populations to identify and isolate high producing cells. Obtaining a host cell line that exhibits a more homogeneous biomanufacturing phenotype would significantly improve the efficiency of the cell line development process. Accordingly, there is a need in the art for host cell lines with improved biomanufacturing capability.

### SUMMARY OF THE DISCLOSURE

The present disclosure is directed to a method of improving specific cellular productivity of a polypeptide of interest in a recombinant cell comprising expressing a nucleic acid encoding the polypeptide of interest in a recombinant cell having a high mitochondrial membrane potential (MMP).

The present disclosure is also directed to a method of improving lactate metabolism in a recombinant cell comprising expressing a nucleic acid encoding the polypeptide of interest in a recombinant cell having a high mitochondrial membrane potential (MMP).

In one aspect, at least 90% of the cells have MMP fluorescence staining intensity of >10³ log as determined by flow cytometry. In one aspect, the dye used in the flow cytometry analysis is Mito-ID, Rh123, DioC6, JC-1, or tetramethyl rhodamine methyl ester (TMRM).

In another aspect, the recombinant cell is a Chinese Hamster Ovary (CHO) cell.

In another aspect, the recombinant cell is comprised in a cell culture. In another aspect, the cell culture is a batch, fed-batch, continuous, or perfusion culture. In another aspect, the cell culture is a fed-batch culture. In another aspect, the recombinant cell is adapted to grow in suspension. In another aspect, the cells are cultured in a bioreactor.

In one aspect, the recombinant cell stably expresses the polypeptide of interest. In another aspect, the polypeptide of interest is an antibody or soluble receptor. In another aspect, the polypeptide of interest is an antibody. In another aspect, the polypeptide of interest is produced at a level of at least 10 pg/cell/day, at least 15 pg/cell/day, at least 20 pg/cell/day, or at least 25 pg/cell/day. In another aspect, the polypeptide of interest is harvested.

In one aspect, the recombinant cells have undergone at least 25, at least 50, at least 75, or at least 100 divisions. In another aspect, the viability of the recombinant cell is increased compared to parental CHO cell cultures containing recombinant cells wherein at least 90% of the cells have MMP fluorescence staining intensity<10³ log as determined by flow cytometry (low MMP). In another aspect, the recombinant cells have increased levels of mGPDH, GAS7, and Mfn2 gene expression compared to the expression level in a control population of recombinant cells, or have been modified to overexpress mGPDH, GAS7, and/or Mfn2.

The present disclosure is also directed to a method of improving cell cloning efficiency of recombinant cells comprising isolating cells having a high level of mitochondrial membrane potential (MMP) and propagating said cells under conditions to promote cell growth. In another aspect, the recombinant cells are CHO cells.

### BRIEF DESCRIPTION OF THE DRAWINGS

FIG. 1 shows generation of an MMP-enriched host. FIG. 1A shows the process of enrichment for a high MMP sub-population using fluorescence activated cell sorting (FACS). The dotted box in the histogram plots represents the sorted population. FIG. 1B shows bivariate plots of green (x-axis) versus orange fluorescence (y-axis) and univariate histogram plots of orange fluorescence depicting Mito-ID dye staining pattern of standard CHO and MMP-enriched hosts.

FIG. 2 shows the phenotype of an MMP-enriched host. FIG. 2A shows two representative confocal microscopic images for each of Mito-ID dye stained standard CHO and MMP-enriched hosts. The orange fluorescence in the cells denotes the energized mitochondria. FIG. 2B shows histogram plots of mitochondrial mass determined by nonyl acridine orange (NAO) staining followed by flow cytometric analysis. FIG. 2C shows flow cytometric analysis of the stability of the MMP trait of the MMP-enriched host over generations. Bivariate plots of green (x-axis) versus orange fluorescence (y-axis) and univariate histogram plots of orange fluorescence depicting Mito-ID dye staining pattern. PDL: Population doubling level.

FIG. 3 shows fed-batch culture characteristics of stable transfectant pools generated from standard CHO and MMP-enriched hosts. Three molecules with varying degrees of difficulty in expression (ETE, DTE1 and DTE2) were chosen. FIGS. 3A-C show integral of viable cell density (IVC) of the pools. FIGS. 3D-F show viability of the pools. FIGS. 3G-I show harvest titer of the pools. FIGS. 3J-L show specific productivity (Qp) of the pools. Data represent mean±SD (n=4). *p<0.05, by two-tailed Student's t-test. ETE, easy-to-express; DTE, difficult-to-express.

FIG. 4 shows the metabolic profile of pools expressing ETE, DTE1 and DTE2 in fed-batch cultures. FIG. 4A-C show glucose consumption and FIG. 4D-F show lactate production of the pools. Data represent mean±SD (n=4).

FIG. 5 shows a representative flow cytometry histogram plots of MMP status of the standard CHO and MMP-enriched host derived stable pools determined by Mito-ID staining.

FIG. 6 shows a characterization of standard CHO and MMP-enriched host derived clones expressing difficult-to-express molecules (DTE1 and DTE2). FIG. 6A shows colony outgrowth in 384-well plates 16 days after single cell sorting. FIG. 6B-C shows harvest titer of the clones after 13 days in fed-batch culture. Each data point represents an individual antibody expressing clone. FIG. 6D-E show aggregate levels determined by high molecular weight (HMW) product analysis. Data in D and E represent mean±SD (n=6). ***p=0.0016, **p=0.004, *p=0.0088, by two-tailed Student's t-test.

FIG. 7A shows a volcano plot showing the expression profile of proteins in standard CHO and MMP-enriched host cells. Dotted line represents the adjusted p value cut off of 5% FDR for quantitation of proteins. Some of the proteins are labeled using their predicted mouse homolog genes. FIG. 7B shows a heat map of differentially expressed proteins was plotted using Perseus software. Z-score (the mean of each row subtracted from each value and the result is divided by the standard deviation) was calculated using the log 2 abundances. The proteins were grouped based on their localization and important functional classes obtained from Ingenuity pathway software (Qiagen). Triplicate data from CHO and MMP-enriched hosts is shown.

FIG. 8 shows a western blot analysis of mitochondria and ER-associated proteins. FIG. 8A shows whole cell lysates and mitochondrial lysates of standard CHO and MMP-enriched host cells were analyzed for proteins involved in mitochondrial function and high MMP phenotype. FIG. 8B-C show whole cell lysates of stable pools generated from standard CHO and MMP-enriched hosts were analyzed for mitochondrial and ER-stress proteins.

FIG. 9 shows bivariate flow cytometry plots of green (x-axis) versus orange fluorescence (y-axis) depicting Mito-ID dye staining pattern of cells expanded from 72 wells isolated following second round of enrichment sort. The wells containing high MMP cells that were selected for generation of MMP-enriched host are shown with red dots.

FIG. 10 shows transgene expression pattern of the stable pools determined by intracellular staining of light and heavy chains using fluorescence-conjugated antibodies.

FIG. 11 shows a scheme for general TMT based quantitative proteomic analysis.

FIG. 12 shows the evaluation of bioreactor fed-batch cell culture performance for parental CHO clones (CDE1a and CDE2a) and MMP-enriched clones (MDE1a and MDE2a) expressing DTE1 and DTE2. FIG. 12A-B Day 14 titers. FIG. 12-C-D Specific productivity (Qp). FIG. 12E-F Viable cell density (VCD). FIG. 12G-H Viability. FIG. 12I-J Glucose consumed. FIG. K-L Lactate produced. Data represent mean±SD (n=2). *p<0.05 by two-tailed Student's t-test.

### DETAILED DESCRIPTION

The present disclosure provides an innovative approach for enriching recombinant host cells with a high mitochondrial membrane potential (MMP). Stable transfectant pools and clonal cell lines expressing difficult-to-express bispecific molecules generated from the MMP-enriched host outperformed the standard host by displaying: (1) improvement in fed-batch productivity, (2) improved lactate metabolism, (3) enhanced long-term cell viability in fed-batch cultures and (4) improved cell cloning efficiency during monoclonal cell line generation. Proteomics analysis together with western blot validation was used to investigate the underlying mechanisms by which high MMP influenced production performance. The MMP-enriched host exhibited multifaceted protection against mitochondrial dysfunction and ER stress.

### I. Definitions

In order that the present disclosure can be more readily understood, certain terms are first defined. As used in this specification, except as otherwise expressly provided herein, each of the following terms shall have the meaning set forth below. Additional definitions are set forth throughout the specification.

It is to be noted that the term “a” or “an” refers to one or more of that entity; for example, “a feed medium,” is understood to represent one or more feed mediums. As such, the terms “a” (or “an”), “one or more,” and “at least one” can be used interchangeably herein.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

The “mitochondrial membrane potential” or “MMP” is a key indicator of mitochondrial activity because it reflects the process of electron transport and oxidative phosphorylation, the driving force behind ATP production. The loss of the MMP is often associated with early stages of apoptosis. The collapse of MMP coincides with the opening of the mitochondrial permeability transition pores, leading to the release of cytochrome C into the cytosol, which in turn triggers other downstream events in the apoptotic cascade. Cellular MMP can be detected using fluorescent dyes that fluoresce in different colors depending upon membrane potential status.

The terms “polypeptide” or “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation of modification, such as conjugation with a labeling component. Also included in the definition are, for example, polypeptides containing one or more analogs of an amino acid (including, for example, unnatural amino acids, etc.), as well as other modifications known in the art. The term “polypeptide” and “protein” as used herein specifically encompass antibodies and Fc domain-containing polypeptides (e.g., immunoadhesins).

As used herein, the term “polypeptide of interest” is used in its broadest sense to include any protein (either natural or recombinant), present in a mixture, for which purification is desired. Such proteins of interest include, without limitation, enzymes, hormones, growth factors, cyotokines, immunoglobulins (e.g., antibodies), and/or any fusion proteins. In some aspects, the protein of interest refers to any protein that can be produced by the methods described herein. In some aspects, the protein of interest is an antibody. In some aspects, the protein of interest is a recombinant protein.

The terms “harvesting,” “purifying,” “separating,” or “isolating,” as used interchangeably herein, refer to increasing the degree of purity of a protein of interest from a composition or sample comprising the protein of interest and one or more impurities. Typically, the degree of purity of the protein of interest is increased by removing (completely or partially) at least one impurity from the composition.

The term “culturing” or “cell culturing” as used herein refers to maintenance or growth of a recombinant cell in a liquid culture medium under a controlled set of physical conditions.

The term “fed-batch culture” or “fed-batch culture process” as used herein refers to a method of culturing cells in which additional components are provided to the culture at some time subsequent to the beginning of the culture process. A fed-batch culture can be started using a basal medium. The culture medium with which additional components are provided to the culture at some time subsequent to the beginning of the culture process is a feed medium. A fed-batch culture is typically stopped at some point and the cells and/or components in the medium are harvested and optionally purified.

As used herein “perfusion” or “perfusion culture” or “perfusion culture process” refers to continuous flow of a physiological nutrient solution at a steady rate, through or over a population of cells. As perfusion systems generally involve the retention of the cells within the culture unit, perfusion cultures characteristically have relatively high cell densities, but the culture conditions are difficult to maintain and control. In addition, since the cells are grown to and then retained within the culture unit at high densities, the growth rate typically continuously decreases over time, leading to the late exponential or even stationary phase of cell growth. This continuous culture strategy generally comprises culturing mammalian cells, e.g., non-anchorage dependent cells, expressing a polypeptide and/or virus of interest during a production phase in a continuous cell culture system.

The term “productivity” or “specific cellular productivity” describes the quantity of a specific protein which is produced by a defined number of cells within a defined time. The specific productivity is therefore a quantitative measure for the capacity of cells to express/synthesize/produce a protein of interest. In the context of industrial manufacturing, the specific productivity is usually expressed as amount of protein in picogram produced per cell and day (pg/cell*day′ or ‘pcd’).

“Recombinant cells” are typically produced by transfecting recombinant DNA into a host cell. Typically, recombinant host cells useful in the methods of the invention are mammalian cells. In one aspect, the recombinant host cells are Chinese Hamster Ovary (CHO) cells.

An “antibody” (Ab) shall include, without limitation, a glycoprotein immunoglobulin which binds specifically to an antigen and comprises at least two heavy (H) chains and two light (L) chains interconnected by disulfide bonds. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region is comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. A heavy chain may have the C-terminal lysine or not. In some aspects, an antibody is a full-length antibody.

An immunoglobulin may derive from any of the commonly known isotypes, including but not limited to IgA, secretory IgA, IgG, IgD, IgE, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, monoclonal and polyclonal antibodies; chimeric and humanized antibodies; human or nonhuman antibodies; wholly synthetic antibodies; and single chain antibodies. A nonhuman antibody may be humanized by recombinant methods to reduce its immunogenicity in man. The term “antibody” can include multivalent antibodies capable of binding more than two antigens (e.g., trivalent antibody). A trivalent antibody are IgG-shaped bispecific antibodies composed of two regular Fab arms fused via flexible linker peptides to one asymmetric third Fab-sized binding module. This third module replaces the IgG Fc region and is composed of the variable region of the heavy chain fused to CH3 with “knob”-mutations, and the variable region of the light chain fused to CH3 with matching “holes”. The hinge region does not contain disulfide bonds to facilitate antigen access to the third binding site. Where not expressly stated, and unless the context indicates otherwise, the term “antibody” includes monospecific, bispecific, or multi-specific antibodies, as well as a single chain antibody.

A “fusion” or “chimeric” protein comprises a first amino acid sequence linked to a second amino acid sequence with which it is not naturally linked in nature. The amino acid sequences which normally exist in separate proteins can be brought together in the fusion polypeptide, or the amino acid sequences which normally exist in the same protein can be placed in a new arrangement in the fusion polypeptide, e.g., fusion of a Factor VIII domain of the disclosure with an Ig Fc domain. A fusion protein is created, for example, by chemical synthesis, or by creating and translating a polynucleotide in which the peptide regions are encoded in the desired relationship. A chimeric protein can further comprises a second amino acid sequence associated with the first amino acid sequence by a covalent, non-peptide bond or a non-covalent bond.

As used herein, “culturing” refers to growing one or more cells in vitro under defined or controlled conditions. Examples of culturing conditions which can be defined include temperature, gas mixture, time, and medium formulation.

As used herein, the terms “expression” or “expresses” are used to refer to transcription and translation occurring within a cell. The level of expression of a product gene in a host cell can be determined on the basis of either the amount of corresponding mRNA that is present in the cell or the amount of the protein encoded by the product gene that is produced by the cell, or both. In some aspects, relative expression refers to the amount of mRNA or protein expressed in a cell relative to a control cell. For example, increased expression can refer to an increase in gene/protein expression in a recombinant cell as compared to a non-transformed cell. In some embodiments, increased expression requires at least 1.5, at least 2.0, at least 2.5, at least 3, at least 4, at least 5, at least 10, at least 50, or at least 100 times the gene/protein expression in a recombinant cell compared to a non-transformed control cell.

Various aspects of the disclosure are described in further detail in the following subsections.

### II. High MMP Recombinant Cells

The present disclosure provides a highly effective approach to increase specific cellular productivity of a polypeptide of interest in a recombinant cell by using cells having a high level of MMP. The present disclosure also provides a highly effective approach to improve lactate metabolism in a recombinant cell by using cells having a high level of MMP.

In one aspect, the disclosure provides a method to isolate a host cell sub-population that exhibits a superior ability for protein production. In one aspect, CHO host cells were stained with an MMP-specific fluorescent dye and enriched for high MMP using fluorescence activated cell sorting (FACS). In one aspect, the dye is Mito-ID, Rh123, DioC6, JC-1, or tetramethyl rhodamine methyl ester (TMRM). In another embodiment, the dye is Mito-ID. High MMP cells were found to have significant improvement in the growth properties, titer, lactate metabolism and subcloning performance compared to the non-clonal pools.

In one aspect, the pool of high MMP recombinant cells is substantially homogenous with respect to MMP. In one aspect, at least 90%, at least 95%, at least 96%, at least 97%, at least 98% or at least 99% of cells express a minimum MMP. In another aspect, at least 90% of high MMP cells have a MMP fluorescence intensity of at least 10³, at least 10⁴, or at least 10⁵ log, as determined by flow cytometry.

In one aspect, the pool of low MMP recombinant cells is substantially homogenous with respect to MMP. In one aspect, at least 90%, at least 95%, at least 96%, at least 97%, at least 98% or at least 99% of cells express a minimum MMP. In another aspect, at least 90% of low MMP cells have a MMP fluorescence intensity of <10³ log, as determined by flow cytometry.

### III. Protein of Interest

In some aspects, the methods disclosed herein can be applied to any protein product (e.g., a protein of interest). In some aspects, the protein product is a therapeutic protein. In some aspects, the therapeutic protein is selected from an antibody or antigen-binding fragment thereof, an Fc fusion protein, an anticoagulant, a blood clotting factor, an engineered protein scaffold, an enzyme, a growth factor, a hormone, an interferon, an interleukin, a receptor, and a thrombolytic. In some aspects, the protein product is an antibody or antigen-binding fragment thereof. In some aspects, the protein is a recombinant protein.

In other aspects, the protein of interest is produced in a host cell. In some aspects, the protein of interest is produced in a culture comprising mammalian cells. In some aspects, the mammalian cells are Chinese hamster ovary (CHO) cells, HEK293 cells, mouse myeloma (NS0), baby hamster kidney cells (BHK), monkey kidney fibroblast cells (COS-7), Madin-Darby bovine kidney cells (MDBK) or any combination thereof. In some aspects, the starting mixture can be a harvested cell culture fluid, a cell culture supernatant, a conditioned cell culture supernatant, a cell lysate, and a clarified bulk.

In some aspects, the proteins produced by the methods described herein are antibodies. Antibodies can include, for example, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelized antibodies, affibodies, Fab fragments, F(ab′) 2 fragments, disulfide-linked Fvs (sdFv), anti-idiotypic (anti-Id) antibodies (including, e.g., anti-anti-Id antibodies), and antigen-binding fragments of any of the above. In some aspects, antibodies described herein refer to polyclonal antibody populations. Antibodies can be of any type (e.g., IgG, IgE, IgM, IgD, IgA or IgY), any class (e.g., IgG1, IgG2, IgG3, IgG4, IgA1 or IgA2), or any subclass (e.g., IgG2a or IgG2b) of immunoglobulin molecule. In some aspects, antibodies described herein are IgG antibodies, or a class (e.g., human IgG1 or IgG4) or subclass thereof. In a aspects, the antibody is a humanized monoclonal antibody. In some aspects, the antibody is a human monoclonal antibody, preferably that is an immunoglobulin. In some aspects, an antibody described herein is an IgG1, or IgG4 antibody.

The growing demand for therapeutic proteins continuously promotes the development of technologies to increase the production of high-quality recombinant proteins in CHO expression systems. Although there has been substantial improvement in the yield of CHO cell-based production processes in recent years, the demands of the ever-expanding highly competitive market still require cells to be more highly productive and to be grown in bioreactors at higher cell densities. In the present disclosure, an innovative approach, based on selection of a cell population with high MMP, led to the establishment of a novel host that inherently exhibits superior attributes for biomanufacturing.

In certain aspects, the MMP-enriched host displayed remarkably improved and consistent production capability for three model antibodies examined. In some aspects, higher IVC, enhanced viability and increased productivity with improved lactate metabolism were observed in the pools generated from the MMP-enriched host. In addition, the clones isolated from the MMP host derived pools demonstrated improved outgrowth in 384-well plates, enrichment of high producer population and improved bioreactor performance. Furthermore, the high MMP phenotype of the host cell line was found to be retained in the recombinant producer pools indicating that the MMP trait is stable and heritable. Therefore, the MMP-enriched host provides a unique tool for generation of highly productive, stable and homogeneous pool populations that will enable enhanced early phase material supply for preclinical toxicology studies and process development activities, thereby accelerating the timelines to the clinic. Increased volumetric and specific productivities for two industrially relevant DTE molecules together with their improved lactate profile demonstrated by the MMP clones in bioreactors suggest that the MMP-enriched host is better equipped to deal with the challenges of developability and manufacture of DTE biotherapeutics. Moreover, given these remarkable improvements in cellular performance, this host will serve as a cost-effective and high-throughput tool for the cell line development process for production of recombinant proteins of various molecular formats.

Mitochondria play a dominant role in production of energy via the oxidative phosphorylation process in the electron transport chain present in the mitochondrial membrane and the functional significance of MMP is multi-faceted. Broadly, MMP is an indicator of mitochondrial function and energy metabolism. Apart from being an essential component in the process of energy storage during oxidative phosphorylation, MMP plays a key role in mitochondrial homeostasis through selective elimination of dysfunctional mitochondria. It is also a driving force for transport of ions (other than H+) and proteins, which are necessary for healthy mitochondrial functioning. Moreover, MMP provides the driving force for ATP synthesis in mitochondria. Due to their enormous energy demand, cells in the production phase require enhanced mitochondrial function to meet their energy need. Although at high MMP the mitochondrial respiratory chain becomes a significant producer of reactive oxygen species and maintaining excessively high mitochondrial MMP is known to be harmful to mitochondria and consequently to the cell, no detrimental effect of high MMP on cell health or production performance was observed.

Comparative proteomics analysis of the MMP-enriched host and the standard CHO host were also performed. After CHO-mouse protein homologs were determined, three pathways were identified that were modulated in the high MMP phenotype and the key proteins involved in these pathways were validated by western blot analysis. Here the MMP-enriched host has up-regulated expression of the Mfn2, PINK1 and p-Parkin proteins. Mfn2 is known to play multiple decisive roles in mitochondrial function, implicating its impact on mitochondrial homeostasis. Mfn2 has been extensively linked to mitochondrial quality control, mainly attributed to its crucial role in mitophagy, with PINK1 and Parkin central to the surveillance mechanism. In this pathway, PINK1 accumulates on defective mitochondria, elicitis the translocation of Parkin from the cytosol and mediates the clearance of damaged mitochondria. Interestingly, the proteomics analysis demonstrated an elevated level of GAS7 in the MMP-enriched host and transfectant pools suggesting a novel functional significance of these proteins in CHO cells that has not been reported to date. Considerable evidence suggests Mfn2 depletion leads to reduced MMP, increased mitochondrial proton leakage and impairment of fatty acid metabolism and oxidative phosphorylation. In addition, Mfn2 forms complexes that are capable of tethering mitochondria to ER, a structural feature essential for mitochondrial energy metabolism, maintenance of intracellular calcium homeostasis and regulation of ER stress response. Mfn2 ablation has been shown to induce ER stress in different models, from mouse tissues to drosophila. In particular, the induction of unfolded protein response (UPR) mediators in Mfn2-deficient mouse embryonic fibroblasts under basal or ER stress conditions and the role of Mfn2 as an upstream modulator of PERK have been reported. In the present disclosure, reduced levels of PERK and the chaperone proteins BiP and PDI in the MMP host derived pools were observed indicating that higher levels of Mfn2 expression are consistent with the maintenance of ER homeostasis with favorable UPR activation and alleviation of ER stress, which most likely contributes to the increased productivity of the pools and clones, particularly for difficult-to-express proteins. Thus, in one aspect the MMP-enriched host has multifaceted protection against mitochondrial dysfunction and ER stress and thereby provides a more favorable intracellular environment for protein production than the standard CHO host.

In one aspect, mitochondrial GPDH, an integral component of the mitochondrial respiratory chain and glycerophosphate (GP) shuttle was upregulated in the MMP-enriched host and transfectant pools. Although specific roles of mGPDH in CHO cells have not been defined, its function at the crossroads of glycolysis, oxidative phosphorylation and fatty acid metabolism in mammalian tissues is known. As the rate limiting component of the glycerophosphate shuttle, mGPDH connects mitochondrial and cytosolic processes and plays a significant role in cellular bioenergetics. Recent findings suggests a role for mGPDH in the regulation of cell growth and mitochondrial metabolism in different types of cancer where mGPDH overexpression was found to be associated with increased growth and oxidative phosphorylation rate. Thus, in one aspect, the high mGPDH phenotype of MMP-enriched host contributes to the increased IVC and enhanced viability of transfectant pools in fed-batch cultures. The consistency of the protein markers observed between the MMP-enriched host cells and the transfectant pools indicate that the FACS-mediated enrichment has enabled the high MMP host to achieve homogeneity and phenotypic stability for the high MMP phenotype.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Examples

### Cell Culture and Stable Transfections

A suspension-adapted, proprietary CHO cell line derived from CHO-K1 and a glutamine synthetase (GS) selection system was used. Stable transfectant pools were generated by nucleofection of linearized expression plasmids and then selected and maintained in proprietary medium supplemented with 75 μM methionine sulfoximine (MSX, Sigma-Aldrich, MO), and 50 mg/L dextran sulfate (Sigma-Aldrich). Suspension cell cultures were grown at 120 rpm on an orbital shaking platform in a humidified incubator set at 37° C. and 6% CO₂. Cells were passaged every 3-4 days. Measurement of viable cell density and viability was accomplished using Trypan Blue and a ViCell automated cell counter (Beckman Coulter, CA).

### Fed-Batch Culture

Antibody production was evaluated by fed-batch culture in 125 mL Erlenmeyer flasks or 96DW plates. The production cultures grew at 35.5° C. in a humidified 6% CO₂ atmosphere for 14 days unless otherwise mentioned. Shaker speed was maintained at 120 rpm for flasks and 350 rpm for 96DW. Cell density and viability were monitored during cultivation in flasks, but not in the 96DW cultures. Proprietary feed was added to the production cultures on days 3, 5, 7, 9, 11 and 13. Antibody titers in the culture supernatant were determined using Protein A biosensors in an Octet QK384 (Pall ForteBio, Fremont, CA) for 96DW and by HPLC (Agilent Technologies, CA) for flask cultures.

### Fed-Batch Culture in Bioreactors

The producer cells were cultured in duplicate 3 L glass stirred-tank bioreactors in fed-batch. Cells were cultured under conditions representative of large-scale manufacturing processes. Temperature of 35.5° C., 300 rpm agitation rate, 50% dissolved oxygen and 7.0±0.1 pH were monitored and regulated using DASGIP controller software (Eppendorf, Germany). Proprietary feed was added to each bioreactor periodically during the run. Fresh cell samples were collected every day for subsequent process analysis and biochemical assays. Offline pH, gases (pCO2 and pO2), lactate, and glucose levels were analyzed using a RAPIDPOINT 500 Blood Gas Analyzer (Siemens, PA). Viable cell density and cell viability were determined using a ViCell. Osmolality and ammonium were measured using a Flex2 (Nova Biomedical, MA). Antibody levels from each bioreactor were quantified using Protein A binding on an HPLC.

### Flow Cytometric Analysis of Mitochondrial Membrane Potential

Status of cellular MMP was analyzed using the dual fluorescent emission dye Mito-ID (Enzo Life Sciences, NY). Cells were stained with Mito-ID dye according to the manufacturer's directions. Briefly, 1×10⁶ cells were harvested by centrifugation at 300×g for 5 minutes, washed with a 1× Mito-ID assay solution and resuspended in Mito-ID detection reagent at a concentration of 1×10⁶ cells/mL. The cells were stained for 30 minutes in the dark at room temperature and analyzed using a BD Symphony cytometer (BD Biosciences, CA). The 488 nm laser was used for excitation, and 525/50 and 582/15 filters were used to acquire green and orange emitted fluorescence, respectively. Bivariate plots of green versus orange fluorescence as well as univariate histogram plots of orange fluorescence were used to analyze MMP. The orange fluorescent population represents energized cells with active MMP. Data analysis was performed using FlowJo software (Tree Star, Inc., OR).

### Cell Sorting

Bulk cell sorting based on MMP for host enrichment and single cell deposition for recombinant clone generation were performed using an Influx cell sorter (BD Biosciences) as described previously.²¹ For the bulk sort, 5×10⁷ cells were harvested by centrifugation. The cells were washed and stained with Mito-ID dye as described above. Based on orange fluorescence intensity, 2×10⁶ cells from gated fractions were deposited in 5 mL collection tubes containing culture medium. The sorted cells were centrifuged, resuspended in 10 mL fresh culture medium and plated in a T-75 flask. Following subculture and expansion, the sorted cells were stained with Mito-ID and the sub-population displaying the peak intensity of MMP staining was isolated using FACS by deposition of five cells per well into individual wells of 96-well plates. For single cell cloning, 1×10⁶ cells were sorted by depositing one cell per well into individual wells of 384-well plates containing conditioned medium. All plates were incubated at 37° C. in a humidified atmosphere with 6% CO₂ for outgrowth.

### Colony Outgrowth Measurement

Confluence per well in 384-well plates was measured using a Cellavista imager (Synentec, Germany) and results analyzed using Nyone software (Synentec, Germany).

### Aggregate Analysis

The fed-batch culture products were captured by analytical Protein A column (POROS A, Thermo Fisher Scientific, MA) using an Agilent 1260 HPLC (Agilent, CA) equipped with a fraction collector. PBS was used for the capturing step and acidified PBS (with 0.1% phosphoric acid) was used for elution. The purified bispecific antibodies were then analyzed by Size Exclusion Chromatography (SEC) for aggregation levels using an ACQUITY UPLC SEC-200 column (Wasters, MA) on the same Agilent 1260 HPLC. The SEC mobile phase was 0.1 M sodium phosphate 0.1M sodium sulfate pH 6.8. The flow rate was set at 0.3 mL/minute and the separation was monitored at 280 nm.

### Determination of Mitochondrial Mass

Mitochondrial mass was determined using nonyl acridine orange (NAO, Thermo Fisher Scientific, MA), a dye that binds to the inner mitochondrial membrane independent of the membrane potential. Cells were incubated at a concentration of 1×10⁶ cells/mL with 500 nM NAO for 10 minutes in the dark at room temperature, washed twice with cold phosphate-buffered saline (PBS) and immediately analyzed on a BD Symphony cytometer using a 488 nm laser for excitation and a 525/50 filter for emission. Data analysis was performed using FlowJo software.

### Confocal Microscopy

All fluorescent images were taken on a LSM880 Airyscanner confocal microscope (Carl Zeiss Inc., Germany). Using 40×1.2NA Water objective, z-stack images of Mito-ID stained cells were acquired from 30 optical sections spaced 0.4 μm apart. Track one used the 488 nm laser with a 495-550 nm emission filter, while track two used a 561 nm laser with a 570-620 nm emission filter.

### Intracellular Staining for Antibody Expression Level

Intracellular expression of the heavy and light chains of antibody molecules was determined by staining cells with fluorescently labeled antibodies specific for heavy or light chains. Briefly, the cells were centrifuged, washed with FACS buffer (1% fetal bovine serum in PBS) and fixed with Fixation Medium A (Thermo Fisher Scientific) for 15 minutes at room temperature. Next, the cells were washed with FACS buffer and stained for 15 minutes at room temperature with the staining solution comprised of goat anti-human IgG (Fc)-Alexa Fluor 488 (Thermo Fisher Scientific) and goat anti-human kappa-APC (Biolegend, CA) in Permeabilization Medium B (Thermo Fisher Scientific). The stained cells were washed and resuspended in FACS buffer before analyzing in a BD Symphony cytometer for the APC and AF488 double positive population. Data analysis was performed using FlowJo software.

### Western Blotting

Mitochondria were isolated using a Mitochondria Isolation Kit (ThermoFisher Scientific, MA) following the manufacturer instructions. Protein extracts were prepared by lysing whole cells and isolated mitochondria in RIPA buffer (ThermoFisher) containing Halt Protease and Phosphatase Inhibitor Cocktail (ThermoFisher) followed by protein concentration measurement using a Pierce BCA assay kit (ThermoFisher). Western blot analysis was performed using a WES system (Protein Simple, CA) with a 12-230 kDa (25 well cartridge) separation module. All samples were diluted to a final loading concentration of 0.4 μg/μl and equal amounts of total protein (2 μg) were loaded for each sample. The instrument settings included separation at 375 V for 25 minutes, blocking for 30 minutes, primary and secondary antibody incubation 30 minutes each and chemiluminescence detection for 15 minutes. The primary antibodies used were rabbit anti-GAS7, PINK1, beta-actin (Abcam, MA), Mfn2, mGPDH (ThermoFisher), phospho-Mfn2 (EMD Millipore, MA), phospho-Ser65-Parkin (Biorbyt, MO), PERK, BiP, PDI (Cell Signaling Technology, MA) and mouse anti-SDHA (Abcam). HRP-conjugated goat anti-rabbit and goat anti-mouse (Abcam) were used as secondary antibodies. Beta-actin and SDHA (succinate dehydrogenase subunit A) were used as loading controls.

### Statistical Analysis

Data are presented as mean±standard deviation (SD). Comparisons of mean differences between groups were made by unpaired two-tailed Student's t-test unless otherwise stated. A probability level of p<0.05 was considered to be statistically significant.

### Proteomics Analysis

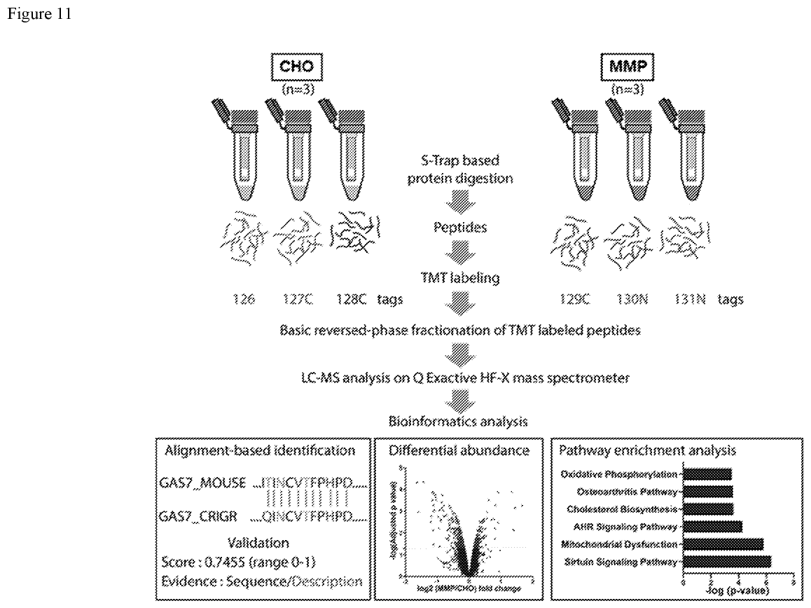

Preparation of proteomics samples: CHO and MMP-enriched host cells were lysed and trypsin digested using S-TRAP. Peptides were labeled with 6-plex TMT (Thermo Fisher Scientific) according to manufacturer's instructions (FIG. 11). LC-MS/MS analysis: Data-dependent acquisition (DDA) LC-MS/MS analyses were carried out on a Q-Exactive HF-X mass spectrometer. Tandem mass spectrometry data were searched against Uniprot Cricetulus griseus database (October 2020) containing 56,400 protein sequences including common laboratory contaminants using Mascot 2.7.0.0 search engine (Matrixscience.com) integrated in Proteome Discoverer (v 2.4, Thermo scientific) software.

### Bioinformatics Analysis

Raw protein abundance values were normalized to the median intensity of each TMT channel. Differential abundance analysis was performed using the Limma Bioconductor package (R version 3.6.0). The volcano plot was created with the ggplot2 R package (R version 3.6.0). Pathway enrichment analysis was performed using Ingenuity Pathway Analysis (IPA) software (Qiagen).

### Sequence Alignment and Machine Learning

Every protein sequence in the Chinese hamster proteome was aligned against every protein sequence in the mouse proteome using the pairwise2 module from the Biopython package (Python version 3.7.4). A logistic regression model was developed to predict functional homology based on percent sequence identity. The model was trained and cross-validated using the scikit-learn Python module (Python version 3.7.4). The trained model was used to assign homologs to each CHO protein.

### Example 1—Generation of a Novel CHO Host Cell Line by MMP Enrichment

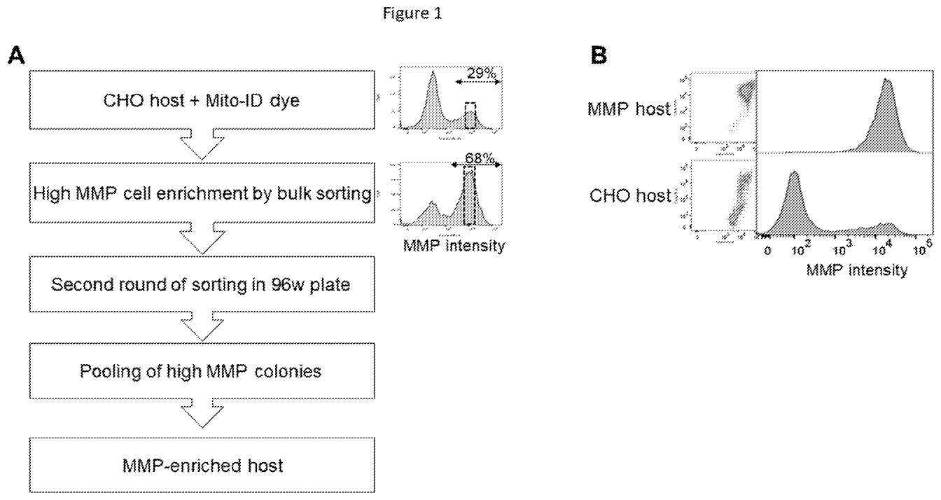

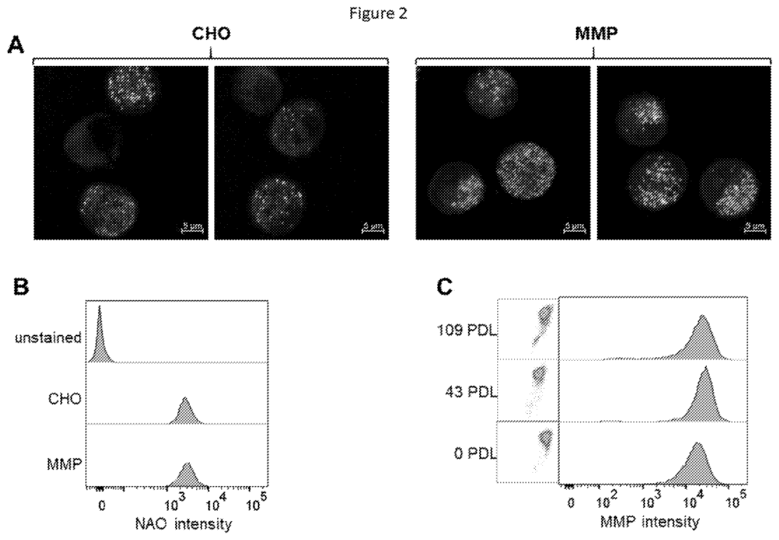

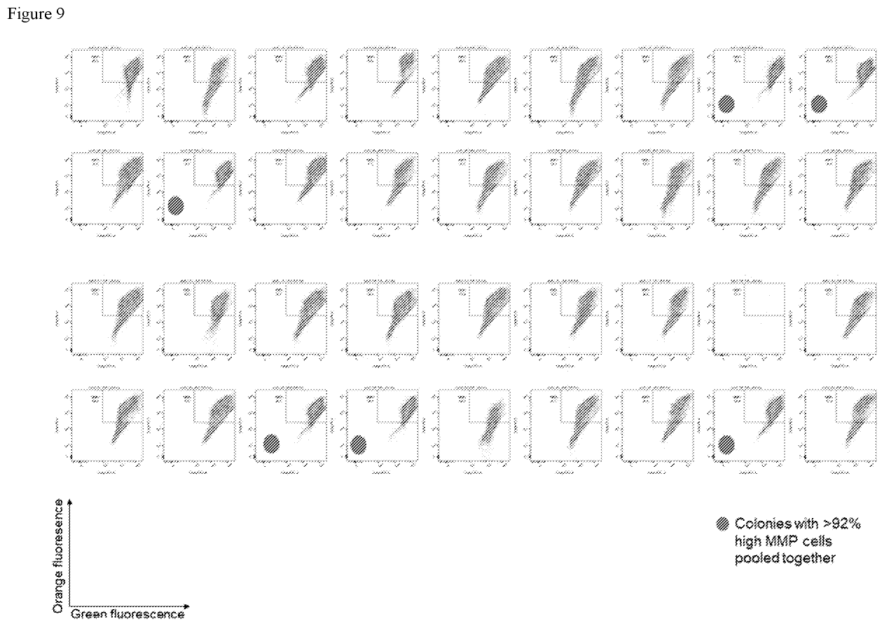

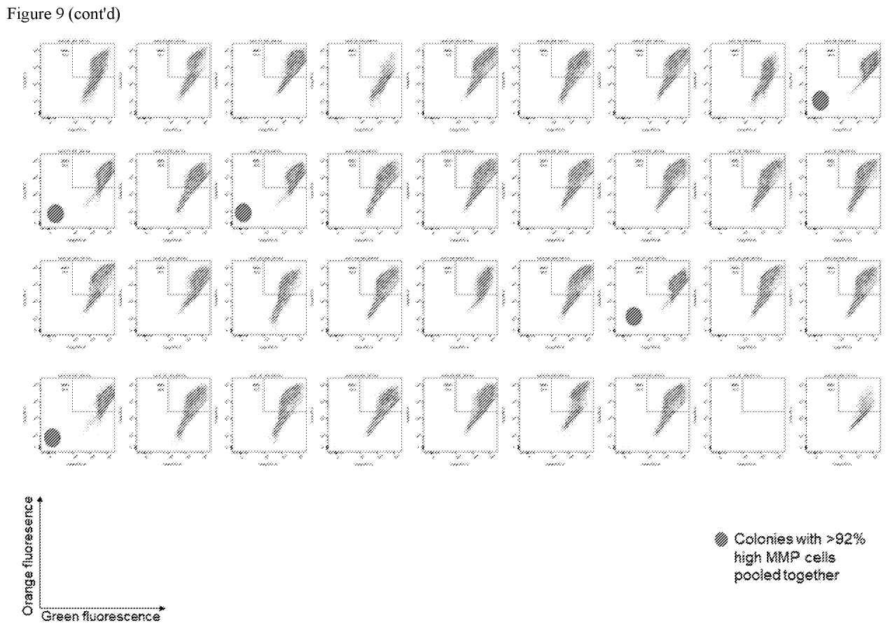

Leveraging the simple flow cytometric staining method for monitoring MMP in living cells, a high MMP sub-population from CHO host cells was isolated and stained with Mito-ID, an MMP dye. Two rounds of sorting by FACS were performed (FIG. 1) to ensure stable enrichment of the host population exhibiting high MMP. In the first step, the sub-population displaying the peak intensity of MMP staining was isolated by bulk sorting and the resulting cells were sub-cultured for two weeks. Following recovery, evaluation of the MMP capacity of the sorted cells showed a partial enrichment of the high MMP population (68% in the sorted cells compared to 29% in the original host cells, FIG. 1A). A second round of sorting was done into a 96-well plate. As before, the sub-population displaying the peak intensity of MMP staining was gated and isolated by depositing five cells per well in a 96-well plate (FIG. 1A). After two weeks, 72 wells with colonies that had reached>20% confluence were scaled-up into wells of a 96-deepwell (96DW) plate and expanded for one week. Evaluation of the 72 cultures displayed varied MMP status ranging between 40% and 96% (FIG. 9). The cultures exhibiting>92% homogeneity of the high MMP phenotype (FIG. 9) were pooled together to generate the MMP-enriched host cell (FIG. 1B). Confocal microscopic imaging of the Mito-ID-stained standard CHO and MMP-enriched hosts validated their respective MMP phenotypes (FIG. 2A). Furthermore, to determine if the MMP enrichment resulted from a difference in the number of mitochondria within each cell, the standard CHO and MMP-enriched hosts were stained with nonyl acridine orange (NAO) dye to measure mitochondrial mass. Flow cytometric analysis revealed no difference in mitochondrial mass per cell among the two host types (FIG. 2B).

To determine the stability of the high MMP trait, the enriched host was continuously expanded for 109 generations (population doubling level, PDL) and their MMP status was evaluated using Mito-ID staining. The enriched population, even after extended culture, maintained a homogeneous high MMP phenotype similar to that of the original pool of enriched colonies (99% at 0 PDL to 95% at 109 PDL, FIG. 2C) suggesting that the enrichment strategy is fundamentally effective in creating a phenotypically stable host cell line. Interestingly, although it is well documented that the inherent plasticity of the CHO nuclear and mitochondrial genome results in continuous genetic and phenotypic drifts in cultured CHO cells with recombinant production load, it may be possible that host cells with no production load have a more stable genome and hence are less likely to undergo phenotypic drift.

### Example 2—Superior Performance of an MMP-Enriched Host in Stable Transfectant Pools

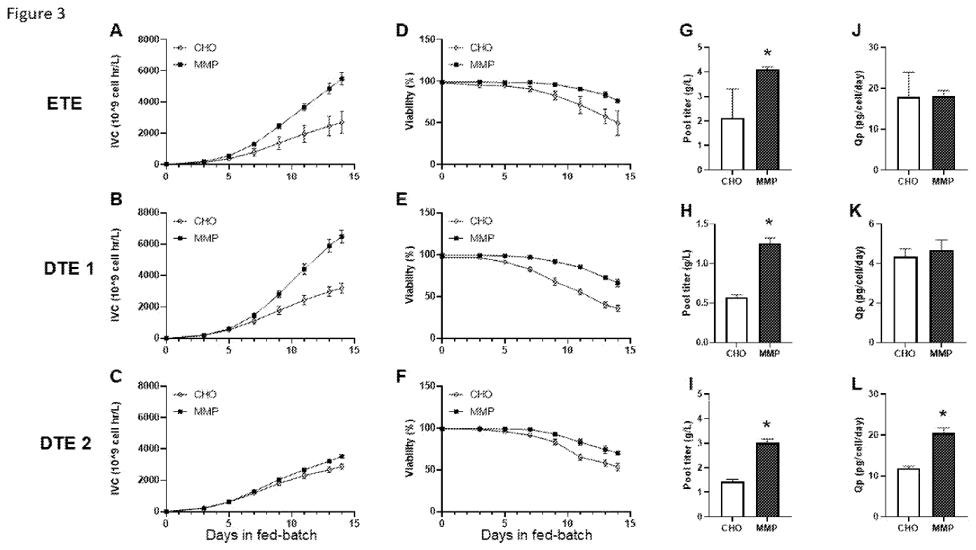

To evaluate the performance of the enriched host, the standard CHO host and the MMP-enriched host were transfected with expression plasmids encoding three different molecules: an easy-to-express mAb (ETE) and two difficult-to-express bispecific antibodies (DTE1 and DTE2). The resulting stable transfectant pools were evaluated for fed-batch productivity in shake flasks. For all three molecules, the MMP pools displayed higher integral of viable cells (IVC) (FIG. 3A-C) and maintained higher viability than the standard CHO pools (FIG. 3D-F). For ETE, MMP-pools had a 1.9-fold increase in final titer compared to CHO pools (FIG. 3G; 2.13 vs. 4.12 g/L, p=0.04). The MMP pools also demonstrated a 2.2 and 2.1-fold increase in titer for DTE1 (FIG. 3H; 0.58 vs. 1.26 g/L, p<0.0001) and DTE2 (FIG. 3I; 1.43 vs. 3.01 g/L, p=0.0005) respectively, compared to the CHO pools. While the specific productivities of ETE and DTE1 remained unchanged between the CHO and MMP pools (FIGS. 3J and 3K), a significant increase in specific productivity was observed for DTE2 pools derived from MMP-enriched host (FIG. 3L, 11.9 vs. 20.5 pg/cell/day, p=0.0034).

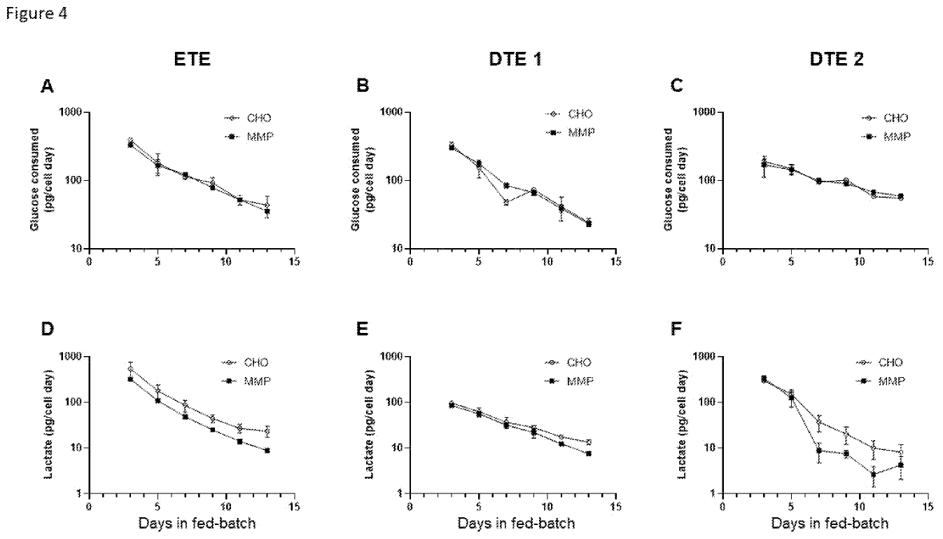

To determine the metabolite turnover during the fed-batch process, we measured the glucose consumption and lactate production (per cell per day) of the pools throughout the culture period. Although glucose consumption was comparable between the MMP and the CHO pools for all three molecules (FIG. 4A-C), lactate production was found to be lower in the MMP pool cultures than the corresponding CHO pools (FIG. 4D-F) suggesting a link between higher mitochondrial activity and improved lactate metabolism. Previously a positive correlation between high mitochondrial membrane potential and lactate consumption rate in CHO cells pointing to a central role of mitochondria on lactate metabolism had been described.

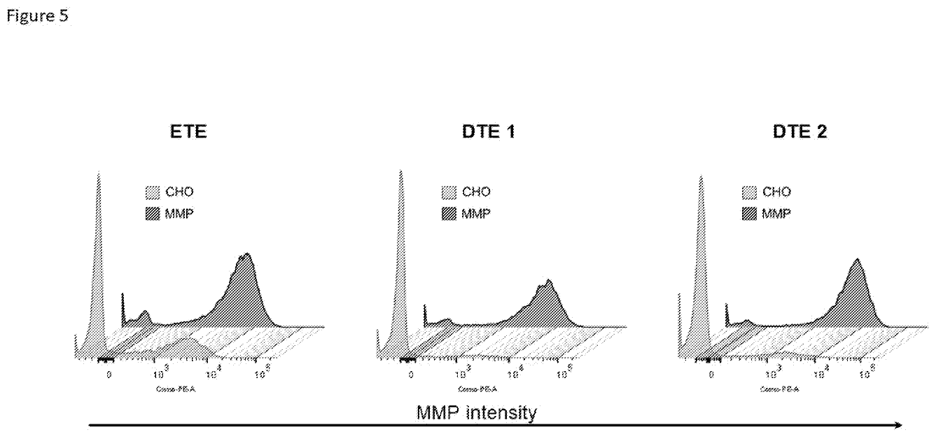

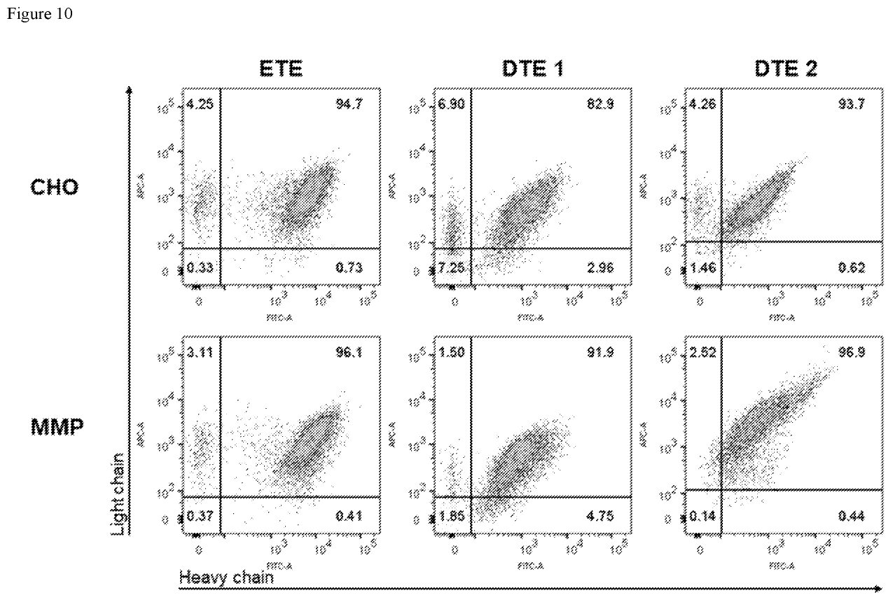

Intracellular heavy chain (HC) and light chain (LC) protein expression in the pools were investigated on the day of last passage before the fed-batch process to assess whether differences in the percentage distribution of expressing populations could be the cause of the difference in productivity observed between the two hosts. For ETE, the analysis revealed similar expression of HC and LC in both hosts, with 95% HC+LC+ for the CHO and 96% for the MMP-enriched host (FIG. 10). For DTE1, the CHO host had a slightly smaller HC+LC+ population compared to the MMP-enriched host (FIG. 10, 83% vs. 92%) and for DTE2, both hosts exhibit similar expression of HC and LC, with 94% HC+LC+ for the CHO (FIG. 10) and 97% for the MMP-enriched host. Therefore, the significant difference in productivity observed between the two hosts is not due to difference in homogeneity of the expressing populations. Furthermore, a comparison of the MMP status of the pools generated from CHO and MMP-enriched host revealed that high MMP phenotype of the host is profoundly maintained in the stable transfectant pools (FIG. 5) thereby further affirming the phenotypic stability of the MMP-enriched host.

### Example 3—Evaluation of MMP-Enriched Host in Cell Line Development Process

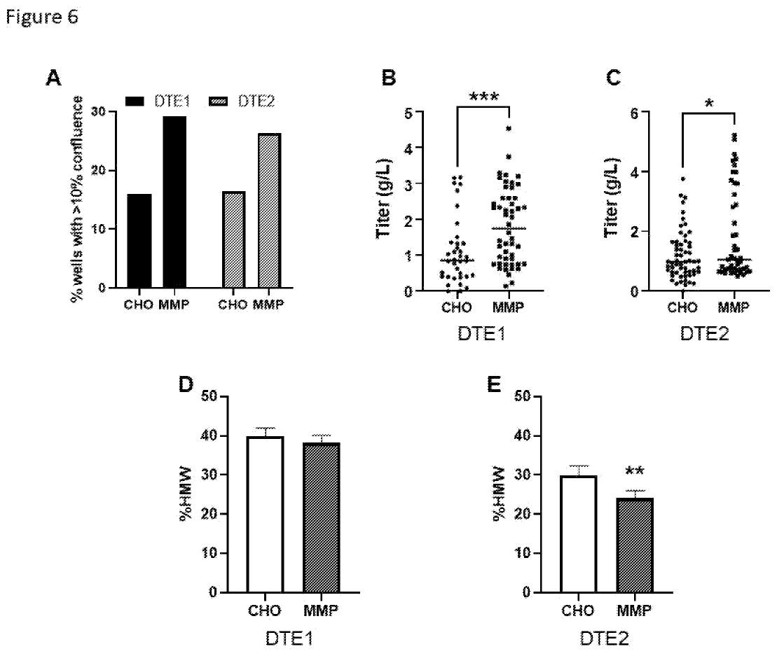

Generation of clonal manufacturing cell lines is a crucial step towards ensuring reproducible product quality for biopharmaceuticals. To determine the suitability and applicability of the new MMP-enriched host for successful biomanufacturing, the performance of clones isolated from the stable pools was evaluated focusing on the difficult-to-express proteins as any improvements have greater potential impacts and benefits for production. First, FACS was used to deposit single cells into 384-well plates from the pools for the DTE1 and DTE2 transfectants generated from the standard CHO and MMP-enriched hosts. Then, the clones were expanded and evaluated in an automated small-scale productivity and product quality screen in a 96DW format. A remarkable improvement was observed in 384-well outgrowth of the clones isolated from the MMP-enriched pools of both molecules when compared with the standard CHO host (16% vs. 29% for DTE1 and 16% vs. 26% for DTE2) (FIG. 6A). In addition, 96DW titer evaluation revealed a significant enrichment of high-producing clones for DTE1 (p=0.0016) and DTE2 (p=0.0088) generated from the MMP-enriched host (FIGS. 6B and 6C). Purified product quality analysis of the fed-batch culture supernatants revealed a significant reduction (p=0.004) in the content of high molecular weight (HMW) species in the DTE2 clones generated from MMP-enriched host with no difference observed for the DTE1 clones (FIGS. 6D and 6E). Taken together, the results suggest that the MMP-enriched host exceeds the standard CHO host in overall performance.

### Example 4—Proteomics Analysis

In order to establish a comprehensive understanding of the different phenotypes of the standard CHO and MMP-enriched hosts their intracellular proteomic profiles were compared. Quantitative proteomic using 24 LC-MS/MS analyses derived from the fractionation of multiplexed tandem mass tag (TMT)-labeled protein digests of CHO and MMP-enriched cells resulted in identification of nearly 8,400 protein groups, which is one of the highest number of CHO cell protein profiles reported to date. Among the proteins identified, 90% were quantitated using isobaric labeling methods. One challenge that was encountered was that the Chinese hamster proteome is not as well annotated as the proteome of model organisms. Therefore, to improve the annotation, proteins were matched in the Chinese hamster and mouse proteomes by their UniProt protein descriptions. For proteins that could not be matched, the percent sequence identity of CHO-mouse protein pairs was calculated and developed a logistic regression model to distinguish homologs from non-homologs. The model was validated via 10-fold cross validation, and the model achieved an accuracy of over 97% on both the training and validation datasets.

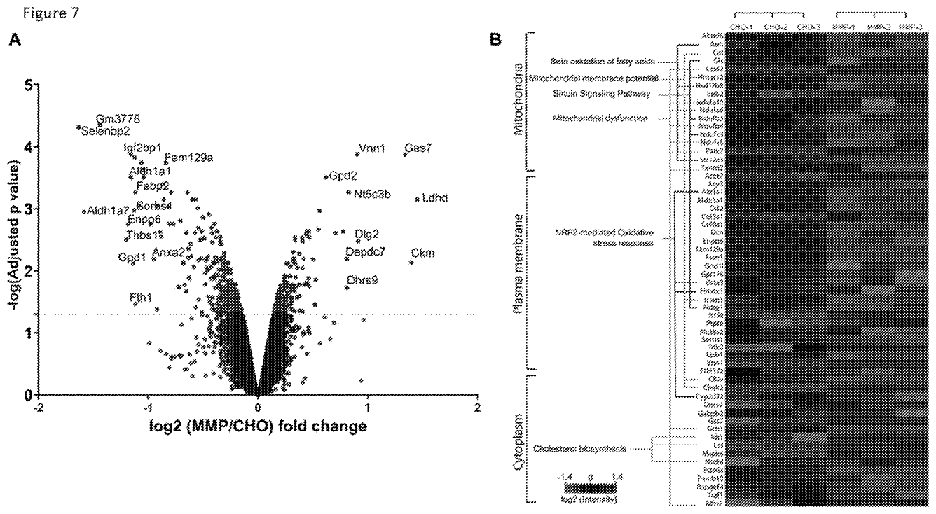

The abundance levels of the proteins involved in mitochondrial function were analyzed and compared between the standard CHO and MMP-enriched hosts. The biological datasets were compared to calculate the “fold-change” and significance by ANOVA and the results were graphed as volcano plots (FIG. 7A). Here, a protein was considered to be differentially abundant if its fold-change was greater than 1.5 and adjusted p-value was less than 0.05. Compared to standard CHO cells, altogether 12 proteins were significantly up-regulated and 44 proteins were significantly down-regulated (by at least 1.5-fold) in the MMP-enriched cells (FIG. 7A). Biological functions associated with the differentially expressed proteins were determined using the Ingenuity pathway functional analysis feature (FIG. 7B). The proteomics dataset from each host type was further annotated using Uniprot GO annotation which includes cellular localization and molecular and biological function. Nearly, 1000 mitochondria associated proteins were found. The Ingenuity pathway analysis (IPA) revealed several of these proteins involved in the regulation of transmembrane potential, depolarization, maintenance of morphology, organization and fusion of mitochondria. The heatmap of differentially expressed proteins from this subset are shown in FIG. 7B.

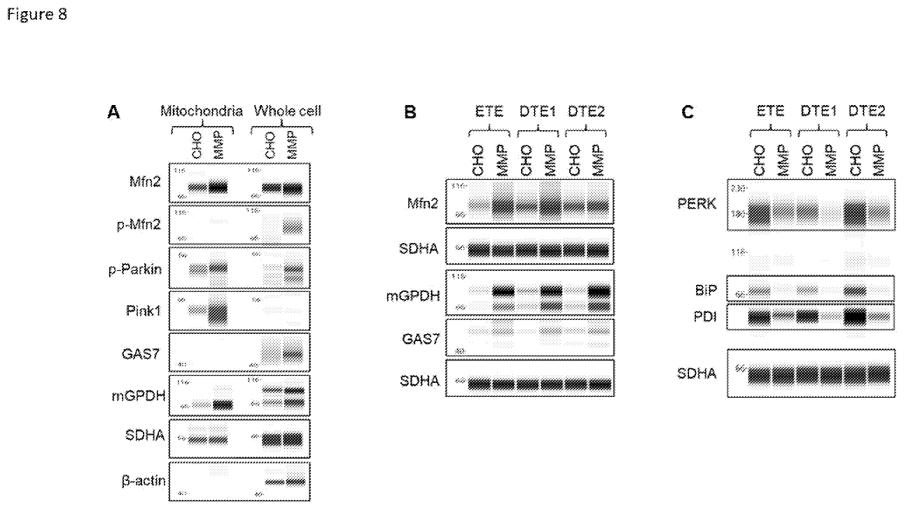

Select findings of the proteomics assays were confirmed by western blotting analysis, focusing on three pathways, namely Mfn2 (mitofusin 2), GAS7 (growth arrest-specific protein 7) and mGPDH (glycerol-3-phosphate dehydrogenase 2) (GPD2) pathways that are involved in mitochondrial function and were found to be upregulated in the MMP-enriched host (FIG. 8A) and transfectant pools (FIG. 8B). Mfn2 plays multiple roles in maintaining a healthy mitochondrial network and hence ensuring proper energetic and metabolic cellular performance. Mfn2 mediates mitochondrial fusion regulates physical contact between mitochondria and ER and maintains mitochondrial quality control by eliminating damaged mitochondria via PINK1/p-Parkin mediated mitophagy. In addition, GAS7 is known to modulate PINK1 function in the regulation of mitochondrial dynamics and metabolism. Indeed, increased expression of p-Mfn2, PINK1 and p-Parkin was observed together with Mfn2 and GAS7 in the MMP-enriched host. Furthermore, several studies have indicated the role of Mfn2 as an ER stress regulatory protein and an upstream modulator of PERK. Ablation of Mfn2 in various cell types has resulted in an up-regulation of ER chaperone proteins both at baseline and under ER stress conditions. Therefore, the standard CHO and MMP-enriched host derived pools were evaluated for expression of PERK and ER chaperone proteins, BiP and PDI using western blot analysis. Reduced levels of PERK, BiP and PDI were observed in the MMP-enriched host-derived pools (FIG. 8C) which is consistent with the inhibitory role of elevated Mfn2 on ER stress noticed in other cell types. Finally, mGPDH, an integral component of mammalian respiratory chain connects mitochondrial and cytosolic processes and plays a critical role in oxidative phosphorylation, lipid metabolism and maintenance of mitochondrial membrane potential of a cell. Thus, it is possible that up-regulation of mGPDH is another mechanism by which the physiological capacity of the MMP-enriched host cells is improved enabling them to deliver superior protein producing pools and clones.

### Example 5—Evaluation of the Performance of the MMP-Enriched Clones in a Bioreactor Environment

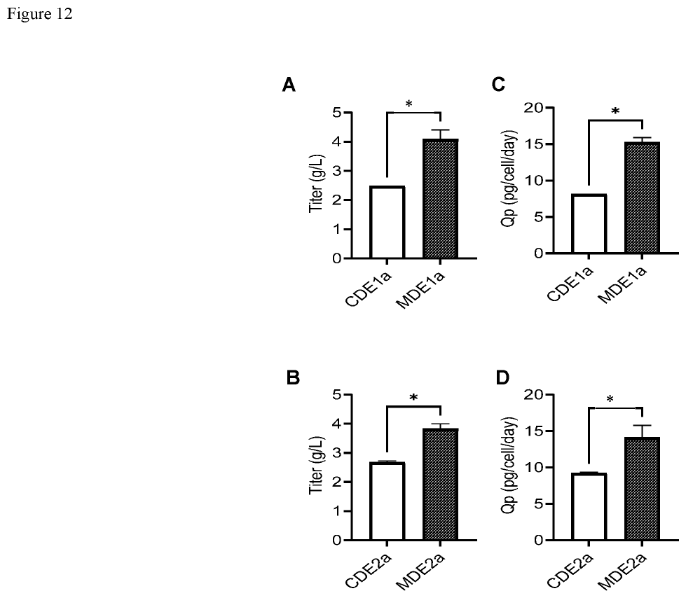

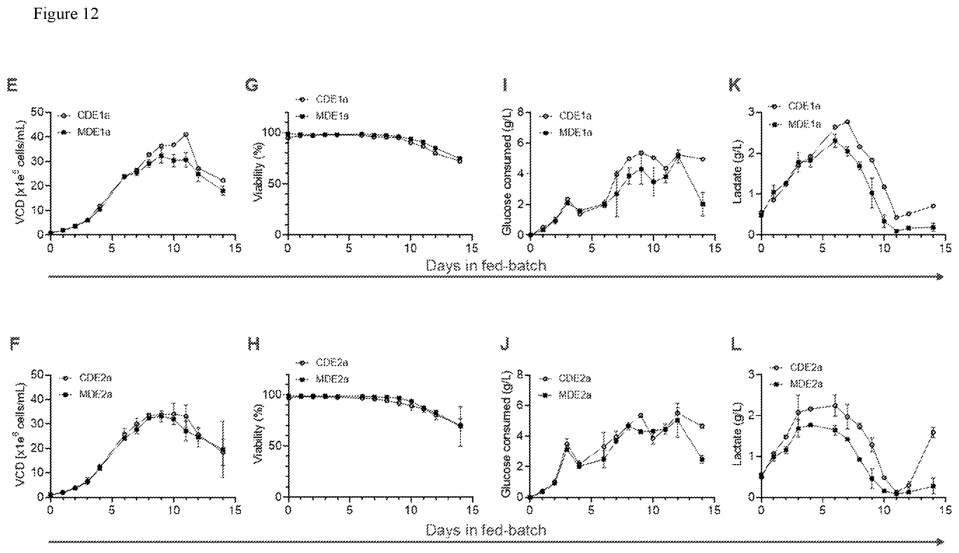

To better understand the improved biomanufacturing capability of the MMP host-derived clones over the standard CHO clones, CDE1a (CHO clone expressing DTE1), CDE2a ((CHO clone expressing DTE2), MDE1a (MMP-enriched clone expressing DTE1) and MDE2a (MMP-enriched clone expressing DTE2) clones were cultured for 14 days in 3 L bioreactors under fed-batch conditions representative of large-scale manufacturing processes. The data demonstrated that MMP host-derived clones MDE1a and MDE2a had a 1.6-fold (2.5 vs 4.1 g/L, p<0.05) and a 1.4-fold (2.7 vs 3.8 g/L, p<0.04) increase in titer, respectively, compared to the corresponding CHO clones (FIG. 12A-B). Similarly, a significant increase in specific productivity was observed for the clones generated from the MMP-enriched host (8.1 pg/cell/day for CDE1a vs 15.2 pg/cell/day for MDE1a, p<0.04 and 9.2 pg/cell day for CDE2a vs 14.1 pg/cell/day for MDE2a, p<0.05) (FIG. 12C-D). While the cell growth pattern (FIG. 12E-F), viability (FIG. 12G-H) and glucose consumption (FIG. 12I-J) remained similar between the CHO and MMP clones, a more favorable lactate profile was observed for both the MMP clones during the course of the bioreactor run (FIG. 12K-L). The standard CHO clones displayed higher lactate production together with an end of run lactate spike (FIG. 12K-L) which reflects a reduced mitochondrial oxidative capacity associated with reduced recombinant protein production in these clones. Taken together, the results suggest that MMP-enriched host is better equipped to deal with the challenges of developability and manufacture of DTE biotherapeutics. Moreover, given these remarkable improvements in cellular performance, this novel host can serve as a cost-effective and high-throughput tool for the cell line development process for production of recombinant proteins of various molecular formats.

What is claimed is:

1. A method of improving specific cellular productivity of a polypeptide of interest in a recombinant cell comprising expressing a nucleic acid encoding the polypeptide of interest in a recombinant cell having a high mitochondrial membrane potential (MMP).

2. A method of improving lactate metabolism in a recombinant cell comprising expressing a nucleic acid encoding a polypeptide of interest in a recombinant cell having a high mitochondrial membrane potential (MMP).

3. A method of improving cell cloning efficiency of recombinant cells expressing a polypeptide of interest comprising isolating cells having a high level of mitochondrial membrane potential (MMP) and propagating said cells under conditions to promote cell growth.

4. The method of any one of claims 1-3, wherein at least 90% of the cells have MMP fluorescence staining intensity>10³ log as determined by flow cytometry.

5. The method of claim 4, wherein the dye used in the flow cytometry analysis is Mito-ID, Rh123, DioC6, JC-1, or tetramethyl rhodamine methyl ester (TMRM).

6. The method of any one of claims 1-5, wherein the MMP level is determined by flow cytometry.

7. The method of any one of claims 1-6, wherein the recombinant cell is a Chinese Hamster Ovary (CHO) cell.

8. The method of any one of claims 1-7, wherein the recombinant cell is comprised in a cell culture.

9. The method of any one of claims 1-8, wherein the cell culture is a batch, fed-batch, continuous, or perfusion culture.

10. The method of claim 9, wherein the cell culture is a fed-batch culture.

11. The method of any one of claims 1-10, wherein the recombinant cell is adapted to grow in suspension.

12. The method of any one of claims 8-11, wherein the cells are cultured in a bioreactor.

13. The method of any one of claims 1-12, wherein the recombinant cell stably expresses the polypeptide of interest.

14. The method of any one of claims 1-13, wherein the polypeptide of interest is an antibody or soluble receptor.

15. The method of claim 14, wherein the polypeptide of interest is an antibody.

16. The method of any one of claims 13-15, wherein the polypeptide of interest is produced at a level of at least 10 pg/cell/day, at least 15 pg/cell/day, at least 20 pg/cell/day, or at least 25 pg/cell/day.

17. The method of any one of claims 1-16, wherein the recombinant cells have undergone at least 25, at least 50, at least 75, or at least 100 divisions.

18. The method of any one of claims 8-17, wherein the cell viability is increased compared to parental CHO cell cultures wherein at least 90% of the cells have MMP fluorescence staining intensity<10³ log as determined by flow cytometry.

19. The method of any one of claims 1-18, further comprising harvesting the polypeptide of interest.

20. The method of any one of claims 1-19, wherein the recombinant cells have increased levels of mGPDH, GAS7, and Mfn2 gene expression or have been modified to overexpress mGPDH, GAS7, and/or Mfn2.
